(19) United States
(12) Patent Application Publication
(10) Pub. No.: US 2021/0198313 A1
(43) Pub. Date: Jul. 1, 2021

(54) METHOD OF PRODUCING IMIDAZOLE DIPEPTIDES

(71) Applicant: Tokai Bussan Co., Ltd., Tokyo (JP)

(72) Inventors: Akira YONEYAMA, Tokyo (JP); Hiroki NAKANISHI, Tokyo (JP); Chiaki SANO, Tokyo (JP)

(73) Assignee: Tokai Bussan Co., Ltd., Tokyo (JP)

(21) Appl. No.: 17/134,185

(22) Filed: Dec. 25, 2020

(30) Foreign Application Priority Data
Dec. 26, 2019 (JP) 2019-235532

(51) Int. Cl. C07K 5/06; C07K 1/18

(52) U.S. Cl. CPC C07K 5/06; C07K 1/18

(57) ABSTRACT

It is an objective of the present invention to provide a method of producing imidazole dipeptides that reduces burden and time for an elution treatment when compared to the prior arts, and furthermore, that can produce high purity imidazole dipeptides with less contamination of creatinine. The above objective is achieved by a method of producing high purity imidazole dipeptides, comprising: (1) subjecting an animal extract containing imidazole dipeptides and creatinine to an ion adsorption treatment in which the animal extract is brought into contact with a strongly acidic cation exchange resin having an alkali metal salt type of ion exchange group at a predetermined pH value to adsorb the imidazole dipeptides onto the resin; and (2) subjecting the strongly acidic cation exchange resin adsorbing the imidazole dipeptides to an elution treatment with the use of an aqueous alkaline solution and at a predetermined pH value to obtain high purity imidazole dipeptides.

### CROSS-REFERENCE TO RELATED APPLICATIONS

Benefit is claimed to Japanese Patent Application No. 2019-235532, filed Dec. 26, 2019, the contents of which are incorporated by reference herein in their entirety.

### TECHNICAL FIELD

The present invention relates to a method of producing imidazole dipeptides.

### BACKGROUND ART

Imidazole dipeptides are dipeptides in which a histidine or histidine derivative having an imidazole group is bounded to an amino acid. Specific examples of imidazole dipeptides include anserine (β-alanyl-1-methylhistidine), carnosine (β-alanyl histidine), valenine (β-alanyl-3-methylhistidine) and homocarnosine (γ-aminobutyryl-L-histidine). Imidazole dipeptides are known to have physiological effects such as anti-fatigue effect and hypoglycemic effect, and have attracted attention as a functional ingredient.

Imidazole dipeptides are produced by methods due to chemical or enzymatic synthesis as well as methods of obtaining imidazole dipeptides from each extract of animals such as fish including tuna, bonito and salmon; mammals including cattle and pig; and birds including chicken.

Among the methods of producing imidazole dipeptides from the animal extract, there are methods with the use of ion exchange treatment. For example, Patent Document 1 discloses a method including the steps of passing the demineralized solution of fish-and-shellfish extract through an H type weakly acidic cation exchange resin to adsorb imidazole dipeptides onto the resin, and then washing the resin with water followed by eluting the imidazole dipeptides with hydrochloric acid and/or brine.

Patent Document 2 also discloses a method including the steps of bringing an animal extract, which contains imidazole dipeptides and free amino acids, into contact with an H type strongly acidic cation exchange resin to adsorb cationic substances contained in the animal extract onto the resin; adjusting pH to the range between 4.5 and 7.5 by adding a first basic solvent to the strongly acidic cation exchange resin with stirring in order to eliminate contaminants; and then eluting the imidazole dipeptides by adding a second basic solvent to the stirred strongly acidic cation exchange resin and by adjusting pH to 7.5 or more.

Patent Document 3 discloses a method including the steps of bringing an animal extract into contact with a strongly acidic cation exchange resin which has been equilibrated to H type in advance using a buffer solution adjusted to the same ranges of electrical conductivity (10±2 mS/cm) and pH (5.0±0.5) as those of the animal extract to adsorb imidazole dipeptides onto the resin, washing the resin with the buffer solution and pure water, and then eluting the imidazole dipeptides by passing an alkaline solution at a pH value in the range between 8 and 12 through the resin or mixing them.

### CITATION LIST

### Patent Documents

[Patent Document 1] Japanese patent gazette 4612549 B

[Patent Document 2] Japanese patent gazette 5512995 B

[Patent Document 3] Japanese patent gazette 5142126 B

### SUMMARY OF THE INVENTION

### Problems to be Solved by the Invention

The method of Patent Document 1 uses an H type weakly acidic ion exchange resin. The adsorption of imidazole dipeptides on the weakly acidic ion exchange resin can be inhibited by salts such as potassium contained in the animal extract. The weakly acidic ion exchange resin is subjected to a rapid change in volume when the type of resin is converted from H type to another ion type, resulting in the problem that the flow pressure loss in the column filled with the resin increases and thus the column may be damaged. As well, the weakly acidic ion exchange resin has a lower specific gravity, and it is difficult to make the resin loosen by means of backwashing. Thus, the method of Patent Document 1 is difficult to use in industrial scale.

To the contrary, each method of Patent Documents 2 and 3 uses a strongly acidic ion exchange resin, which prevents the adsorption of imidazole dipeptides onto the ion exchange resin from being inhibited due to salts. However, the method of Patent Document 2 is characterized by the increased elution efficiency of imidazole dipeptides by eluting imidazole dipeptides at the two separate pH ranges using the two different elution solvents. Thus, the method of Patent Document 2 may provide the significant burden and time of elution treatment, thereby causing the economic efficiency to become inferior, and causing the yield to become reduced.

The method of Patent Document 2 is also directed to remove arsenobetaine which is an arsenic compound commonly found in the fish and shellfish extract, and nowhere is a disclosure relating to creatinine in Patent Document 2. In fact, the present inventors have found that when the chicken extract was subjected to an adsorption treatment with an H type strongly acidic cation exchange resin and an elution treatment with an alkaline solution, the content of creatinine relative to imidazole dipeptides was 33.4% by mass. This is not shown in FIGS. 1 and 2 of Patent Document 2. Therefore, the removal of creatinine cannot be almost achieved with the method of Patent Document 2.

Animal extracts rich in imidazole dipeptides, such as chicken breast meat extract, contain large amounts of proteins, free amino acids, and inorganic salts as well as imidazole dipeptides, and in particular, the chicken breast meat extract contains creatinine in the same or similar molar amount as imidazole dipeptides. Creatinine is a waste product which is produced in a body of animal as a metabolic product of creatine phosphate, which is a source of energy for muscles, and is eliminated from the body by the kidney function. Therefore, when the imidazole dipeptides are extracted from the animal extract, creatinine contained in the resulting imidazole dipeptides is regarded as an impure substance.

As shown in the GPC-HPLC chromatogram described in Patent Document 3, the ratio of peak height of creatinine relative to that of imidazole dipeptides tends to increase in the cation exchange treatment solution (FIG. 2) compared to the raw material (FIG. 1), and thus the method of Patent Document 3 is very low in the removal efficiency of creatinine coexisting with imidazole dipeptides. Therefore, the method of Patent Document cannot provide high purity imidazole dipeptides since the contaminants are adsorbed and eluted together with imidazole dipeptides.

In view of the above circumstances, it is an objective of the present invention to provide a method of producing imidazole dipeptides, which results in reduced burden and time for an elution treatment when compared to the method of Patent Document 2, and allows to obtain high purity imidazole dipeptides with less creatinine when compared to the method of Patent Document 3.

### Means for Solving the Problems

In the course of extensive efforts to find a way to solve the above-identified problems, the present inventors came to think that if an H type strongly acidic cation exchange resin was employed for the separation and purification of imidazole dipeptides, protons might be released when imidazole dipeptides and inorganic salts contained in an animal extract were adsorbed to the resin, resulting in a decrease in pH around the resin. Furthermore, it was assumed that in this case, weak electrolytes such as creatinine, proteins and colored substances in the animal extract might turn to be positively charged and be easily adsorbed to the resin, resulting in a decrease in purity of imidazole dipeptides obtained as eluted substances. Finally, the present inventors found that when the animal extract containing imidazole dipeptides was brought into contact with a strongly acidic cation exchange resin of alkali metal salt type such as Na type, but not H type, a drop in pH at equilibrium adsorption prevented creatinine from being adsorbed to the resin, and allowed imidazole dipeptides to be efficiently adsorbed to the resin. Furthermore, in the case of using the alkali metal salt type resin, the adsorbed ion selectivity is higher than that of the H type resin, and the pH value at the time of adsorption is kept around neutral so that cationic contaminants contained in the animal extract can be effectively removed during the adsorption treatment.

The present inventors also explored possibilities of separating imidazole dipeptides from creatinine based on the difference in pH (dissociation constant) at adsorption equilibrium between them during the adsorption onto the strongly acidic cation exchange resin. By focusing on the charge states of imidazole dipeptides and creatinine, the present inventors experimentally confirmed the pH at which imidazole dipeptides had a positive charge and the positive charge of creatinine was weakened. The present inventors also found that the equilibrium adsorption under the condition could increase the exclusivity of creatinine and obtain high purity imidazole dipeptides. In particular, it was found that imidazole dipeptides were preferentially adsorbed over creatinine in a predetermined range of pH when an alkali metal salt type strongly acidic cation exchange resin was used.

Based on these findings, the present inventors set pH of the animal extract to a predetermined range, and executed an ion adsorption treatment using a strongly acidic cation exchange resin having an alkali metal salt type ion exchange group. Surprisingly, it was found that high purity imidazole dipeptides with a yield sustained and a creatinine content reduced could be obtained without the stepwise elution treatments as described in Patent Document 2. More surprisingly, the obtained imidazole dipeptides had a smaller content of creatinine, an improved coloration, and a reduced bitterness, as compared to those obtained by the method of Patent Document 3.

Finally, on the basis of the above findings, the present inventors have successfully invented a method of producing high purity imidazole dipeptides, including the steps of subjecting an animal extract to an ion adsorption treatment carried out in a predetermined range of pH and with the use of a strongly acidic cation exchange resin having a alkali metal salt type ion exchange group to preferentially adsorb the imidazole dipeptides; and subjecting the adsorbed imidazole dipeptides to an elution treatment with the use of an aqueous alkaline solution to obtain high purity imidazole dipeptides. Such as, the present invention has been completed on the basis of the findings and successful examples that were first found or obtained by the present inventors.

According to the present invention, there is provided a method in the following aspects:

[1] A method of producing high purity imidazole dipeptides, comprising:

(1) subjecting an animal extract containing imidazole dipeptides and creatinine to an ion adsorption treatment in which the animal extract is brought into contact with a strongly acidic cation exchange resin at a pH value so that the imidazole dipeptides are adsorbed onto the strongly acidic cation exchange resin, wherein the pH value is a pH value at which the imidazole dipeptides become positively charged and the ratio of the positive charge of creatinine to the positive charge of imidazole dipeptides becomes equal to or less than 20%, and the strongly acidic cation exchange resin has an alkali metal salt type of ion exchange group; and

(2) subjecting the strongly acidic cation exchange resin adsorbing the imidazole dipeptides to an elution treatment with the use of an aqueous alkaline solution and at a pH value which enables the imidazole dipeptides to become charged at zero or negatively charged to obtain high purity imidazole dipeptides.

[2] The method according to [1] above, wherein the step (1) is subjecting an animal extract containing imidazole dipeptides and creatinine to an ion adsorption treatment in which the animal extract is brought into contact with a strongly acidic cation exchange resin at a pH value followed by subjecting the strongly acidic cation exchange resin to a washing treatment using water so that the imidazole dipeptides are adsorbed onto the strongly acidic cation exchange resin, wherein the pH value is a pH value at which the imidazole dipeptides become positively charged and the ratio of the positive charge of creatinine to the positive charge of imidazole dipeptides becomes equal to or less than 20%, and the strongly acidic cation exchange resin has an alkali metal salt type of ion exchange group.
[3] The method of any one of [1] to [2] above, wherein in the step (1), the pH value is in the range between 5.6 and 8.2, and/or in the step (2), the pH value is in the range between 8.5 and 15.0.
[4] The method according to any one of [1] to [3] above, wherein the strongly acidic cation exchange resin is a strongly acidic cation exchange resin having an ion exchange group converted to an alkali metal salt type by means of passing an aqueous acid solution and an aqueous alkali metal salt solution in sequence through the resin.
[5] The method according any one of [1] to [4], wherein the aqueous alkaline solution is an aqueous alkali metal hydrate solution.
[6] The method according to any one of [1] to [4] above, wherein the aqueous alkaline solution is an aqueous sodium hydroxide solution.
[7] The method according to any one of [1] to [6], wherein the alkali metal salt is at least one alkali metal salt selected from the group consisting of sodium and potassium.
[8] The method according to any one of [1] to [7], wherein the animal extract is an animal extract subjected to a demineralization treatment.
[9] The method according to any one of [1] to [8], wherein the animal extract is at least one animal extract selected from the group consisting of chicken extract, bovine extract, pig extract, salmon extract, bonito extract and tuna extract.

### Effect of the Invention

The method according to one embodiment of the present invention enables an elution treatment to be carried out with no significant burden or time by using a strongly acidic cation exchange resin even if any complicated facilities, equipments, operations and the like are not applied. Based on the method according to one embodiment of the present invention, it is also probable to obtain high purity imidazole dipeptides with a reduced level of creatinine. Therefore, the method according to one embodiment of the present invention is a simple and economical method, and thus the method can be carried out on an industrial scale.

Furthermore, by employing the method according to one embodiment of the present invention, imidazole dipeptides can be recovered from an animal extract in an amount equal to or more than that of the method of Patent Document 3. As such, the method according to one embodiment of the present invention can reduce the ratio of creatinine relative to imidazole dipeptides (creatinine/imidazole dipeptides) as compared to the method of Patent Document 3. Furthermore, by employing the method according to one embodiment of the present invention, it is possible to obtain highly palatable imidazole dipeptides with color improved and bitterness reduced.

### BRIEF DESCRIPTION OF THE DRAWINGS

FIG. 1 is a graph illustrating the relationship between pH and effective charge with respect to each of anserine, carnosine and creatinine, as described in Examples below.

FIG. 2 is a graph illustrating the relationship between pH and the positive charge ratio of creatinine to imidazole dipeptides, as described in Examples below.

FIG. 3 is a graph illustrating the relationship between pH at the time of adsorption and the ratio of the amount adsorbed onto the resin relative to the amount loaded, as described in Examples below.

FIG. 4 is a graph illustrating changes in pH, Brix and the content of imidazole dipeptides relative to the flow rate of chicken extract, as described in Examples below.

FIG. 5A are chromatograms illustrating the measured results of GPC-HPLC demonstrating the contents of imidazole dipeptides and creatinine in the chicken extract as a raw material and the ion exchange eluate obtained by carrying out the method according to one embodiment of the present invention, as described in Examples below.

FIG. 5B are chromatograms corresponding to FIG. 1 and FIG. 2 of Patent Document 3.

FIG. 6 are chromatograms illustrating the measured results of GPC-HPLC demonstrating the contents of imidazole dipeptides and creatinine in the salmon extract as a raw material and the ion exchange eluate obtained by carrying out the method according to one embodiment of the present invention, as described in Examples below.

### DESCRIPTION OF EMBODIMENTS

While a method that forms one embodiment of the present invention will now be described in detail, the present invention may take various embodiments to the extent that its objective can be achieved.

Unless otherwise specified, each term used herein is used in the meaning commonly used by those skilled in the art and should not be construed to have any meaning that is unduly limiting. Also, any speculations and theories herein are made on the basis of the knowledge and experiences of the present inventors and as such, the present invention is not bound by any such speculations and theories.

The term “RV” means a multiple number of flow rate of solvent relative to an amount of resin. For example, if the two times amount of animal extract relative to the amount of resin is passed through the resin, RV makes 2.

The term “SV” is Space Velocity, which means a ratio per hour of a liquid amount flowed (volume) to a resin amount (volume). For example, if 5 m³ of liquid is passed through 1 m³ of resin for an hour, SV makes 5.

The term “and/or” as used herein means either any one of, any combination of two or more of, or combination of all of listed related items.

The wording “to” for indicating a range of values is intended to include values preceding and following the wording; for example, “0 wt % (% by mass) to 100 wt %” means a range from 0 wt % or more and 100 wt % or less.

The terms “include,” “comprise,” and “contain” mean that an element(s) other than an element(s) as explicitly indicated can be added as inclusions, which are, for example, synonymous with “at least include,” but encompasses the meaning of “consist of” and “substantially consist of”. In other words, the terms may mean, for example, to include an element(s) as explicitly indicated as well as any one element or any two or more elements, to consist of an element(s) as explicitly indicated, or to substantially consist of an element(s) as explicitly indicated. Such elements include limitations such as components, steps, conditions, and parameters.

The number of digits of an integer equals to its significant figure. For example, 1 has one significant figure and 10 has two significant figures. For a decimal number, the number of digits after a decimal point equals to its significant figure. For example, 0.1 has one significant figure and 0.10 has two significant figures.

### Summary of Method According to One Embodiment of the Present Invention

A method according to one embodiment of the present invention relates to a method of producing imidazole dipeptides from an animal extract by using an ion exchange treatment. The method according to one embodiment of the invention includes the following steps (1) and (2):

(1) subjecting an animal extract containing imidazole dipeptides and creatinine to an ion adsorption treatment in which the animal extract is brought into contact with a strongly acidic cation exchange resin at a pH value so that the imidazole dipeptides are adsorbed onto the strongly acidic cation exchange resin, wherein the pH value is a pH value which the imidazole dipeptides become positively charged and the ratio of the positive charge of creatinine to the positive charge of imidazole dipeptides becomes equal to or less than 20%, and the strongly acidic cation exchange resin has an alkali metal salt type of ion exchange group; and

(2) subjecting the strongly acidic cation exchange resin adsorbing the imidazole dipeptides to an elution treatment with the use of an aqueous alkaline solution and at a pH value which enables the imidazole dipeptides to become charged at zero or negatively charged to obtain high purity imidazole dipeptides.

The imidazole dipeptides are not particularly limited as long as they are those as normally known. For example, the imidazole dipeptide can be said to be a dipeptide in which a histidine or a histidine derivative having an imidazole group is bound to an amino acid. Specific examples of imidazole dipeptides include anserine (β-alanyl-1-methylhistidine), carnosine (β-alanylhistidine), balenine (β-alanyl-3-methylhistidine) and homocarnosine (γ-aminobutyryl-L-histidine).

The animal extract may be obtained by dissolving components contained in meats and other parts of fishes, birds, mammals and other animals in an extracting medium. The type of animal is not particularly limited as long as it is an animal that contains imidazole dipeptides in its meats or other body parts. Examples of animal include bonito, tuna, salmon, eel, shark, cattle and chicken that contain anserine in high content; pig that contains carnosine in high content; and whales that contains balenine in high content. The animal extract is preferably derived from meats, e.g., muscles from livestock animals such as chicken, cattle and pig and fishes such as salmon, bonito and tuna, since the muscles contain a large content of imidazole dipeptides, and the animals are abundant in terms of resources, or are easy to breed.

The method of obtaining an animal extract is not particularly limited. The animal extract may be obtained by subjecting the animal parts containing imidazole dipeptides to known extraction methods such as a water extraction, a hot water extraction, an alcohol extraction and a supercritical extraction, or may be commercially available. The animal extract may be obtained by subjecting the resulting extract from the above extraction methods to processing treatments such as a solid-liquid separation treatment, a concentration treatment, a drying treatment and a dilution treatment. The animal extract can contain insoluble solids and fats which may cause problems that prevent imidazole dipeptides from being adsorbing onto a strongly acidic cation exchange resin and that give rise to a deterioration of the strongly acidic cation exchange resin. Thus, it is preferable to remove the insoluble solids and fats contained in the animal extract, e.g., by using the above processing treatments or other treatments.

The animal extract is preferred to be subjected to a demineralization treatment since in the subsequent ion exchange treatment, the yield loss may be decreased and the amount of imidazole dipeptides adsorbed per resin may be improved, resulting in a higher purity of imidazole dipeptides. For example, the demineralization treatment is preferably carried out using an electrodialysis demineralization device “DW-3E2 type” (manufactured by AGC Engineering) equipped with CMV-N/AMV-N as a cation exchange membrane/anion exchange membrane under the condition in which the target conductivity per 1% by mass of imidazole dipeptides is in the range between 2 mS/cm and 14 mS/cm, preferably about 5 mS/cm.

### [Step (1): Adsorption Treatment Step]

In Step (1), an animal extract is brought into contact with a strongly acidic cation exchange resin having an alkali metal salt type ion exchange group at a predetermined pH to adsorb imidazole dipeptides onto the strongly acidic cation exchange resin.

The cation exchange resin is an ion exchange resin having a cationic ion exchange group. The cation exchange resins are broadly divided into two types: strongly acidic cation exchange resins having a strongly acidic ion exchange group such as a sulfonic acid group, and weakly acidic cation exchange resins having a weakly acidic ion exchange group such as a carboxylic acid group. Among them, Step (1) employs a strongly acidic cation exchange resin, preferably a strongly acidic cation exchange resin having a sulfonic acid group as an ion exchange group.

The strongly acidic cation exchange resin may be produced by known methods or commercially available. Examples of the strongly acidic cation exchange resin as being commercially available include the strongly acidic cation exchange resins identified with the following brand names: “DIAION” (Mitsubishi Chemical); “Amberlite” (Organo); “Dowex”, “Muromac” and “Levacit” (Muromachi Chemical). Specific examples of the strongly acidic cation exchange resin include “DIAION SK1B” (degree of cross-linking, 8%), “Amberlite IR-120B”, “Dowex HCR-S”, “Muromac C101”, and “Levacit S1668”.

At the time when contacting with the animal extract, the ion exchange group of the strongly acidic cation exchange resin is in the form of alkali metal salt type. For the purpose, if the ion exchange group has been already in the form of alkali metal salt type, the strongly acidic cation exchange resin may be used as it stands. On other occasions, for example, when being in the form of H type, the ion exchange group is converted to the form of alkali metal salt type. The method of converting an ion exchange group to the form of alkali metal salt type is not particularly limited. Examples of the method include a method including converting an ion exchange group of a strongly acidic cation exchange resin to the form of H type with the use of acid, and then converting the resulting H type ion exchange group to be in the form of alkali metal salt type by a treatment in which the resin is immersed in a solution containing an alkali metal salt, or the solution is passed though the resin.

While the form of alkali metal salt type is not particularly limited, for example, the form includes Na type, K type and Li type. Among them, the forms of Na type and K type are preferably employed since the ion exchange group in the forms can be easily and economically obtained. For the purpose of obtaining a strongly acidic cation exchange resin having a Na type or K type ion exchange group, salts may be used, and such salts include neutral salts such as sodium chloride and potassium chloride; hydroxides such as sodium hydroxide, potassium hydroxide, sodium bicarbonate and sodium carbonate. In addition, if the obtained high purity imidazole dipeptides are used for foods, the alkali metal salt type is preferably Na type. For the conversion to the form of Na type, it is preferable to use an aqueous solution of sodium chloride, sodium hydroxide or their combination in view of being generally used and economic efficiency.

For example, the conversion of a strongly acidic cation exchange resin to the form of Na type may be achieved by passing a 0.5 N to 2 N aqueous hydrochloric acid solution through a column filled with a strongly acidic cation exchange resin at a flow rate of 1 RV to 4 RV to convert the resin to the form of H type since the exchange capacity of the strongly acidic cation exchange resin is 2 eq/L, and then passing a 0.5 N to 2 N aqueous sodium hydroxide solution through the column at a flow rate of 1 RV to 4 RV or passing a 3% by mass to 12% by mass aqueous sodium chloride solution at a flow rate of 1 RV to 4 RV to convert the resin to the form of Na type.

In the course of converting a strongly acidic cation exchange resin to the form of alkali metal salt type, it may be able to convert the resin to the form of Na type without converting the resin to the form of H type in advance, for example, by subjecting an animal extract to a demineralization treatment in advance or by passing a large volume of aqueous alkali metal salt solution through the resin.

If the animal extract is in the solid form, for example, in the powder form, or in the concentrated state, the animal extract is dissolved or diluted in water to form an aqueous solution. If the animal extract has been already in the form of aqueous solution, the animal solution can be used as it stands. In order to reduce the inhibitory effect of contaminants on the adsorption of imidazole dipeptides onto the strongly acidic cation exchange resin, Brix per 1% by mass of imidazole dipeptides of the animal extract is preferably in the range between 6.0% and 8.0%, more preferably about 7.5% while the electrical conductivity of the animal extract is preferably in the range between 5 mS/cm and 15 mS/cm, more preferably equal to or less than 13 mS/cm.

The method for bringing the animal extract into contact with the strongly acidic cation exchange resin is not particularly limited, as long as the imidazole dipeptides contained in the animal extract can be adsorbed onto the strongly acidic cation exchange resin. Examples of the method include a batch method including immersing the strongly acidic cation exchange resin in the animal extract, and a column method including passing the animal extract through a column packed with the strongly acidic cation exchange resin. While as a specific example the column method for bringing the animal extract into contact with the strongly acidic cation exchange resin will now be described, the present invention is not limited to the specific example.

The contact between the animal extract and the strongly acidic cation exchange resin is carried out at a pH value which enables the imidazole dipeptides contained in the animal extract to become positively charged, and enables the ratio of the positive charge of creatinine to the positive charge of imidazole dipeptides to become equal to or less than 20%. In other word, the contact is carried out in a manner that the animal extract is flowed through the column packed with the strongly acidic cation exchange resin and the pH value in the column immediately reaches to a value such that the imidazole dipeptides contained in the animal extract get to be positively charged and the ratio of the positive charge of creatinine to the positive charge of imidazole dipeptides gets to be equal to or less than 20%.

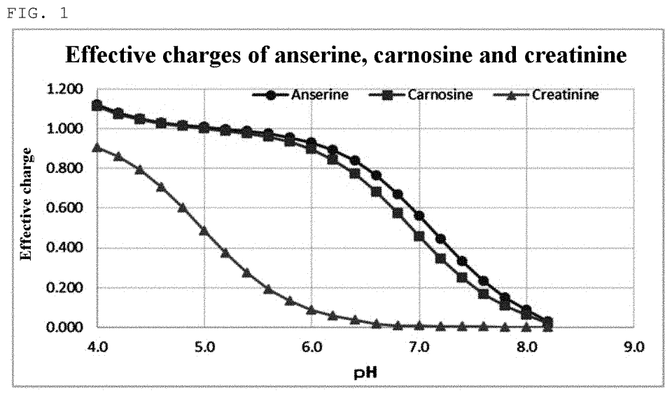

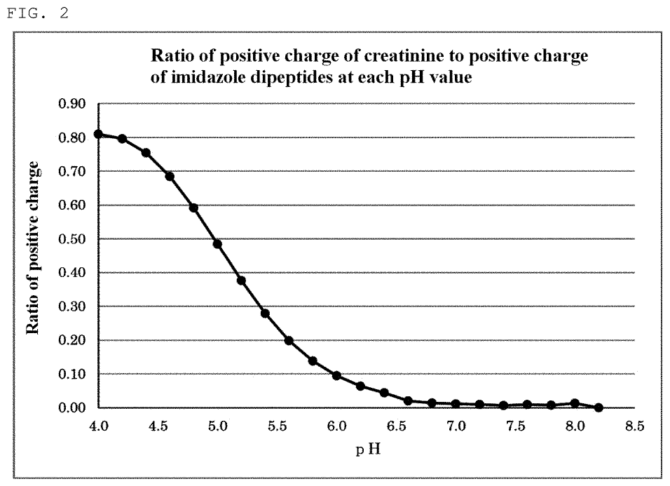

As shown in FIG. 1, the pH value at which imidazole dipeptides such as anserine and carnosine become positively charged is up to about 8.2. As shown in FIG. 2, the pH value at which the ratio of the positive charge of creatinine to the positive charge of imidazole dipeptides is equal to or less than 20% is about 5.6 or higher. From above, the contact between the animal extract and the strongly acidic cation exchange resin is preferable to be carried out at a pH value in the range between 5.6 and 8.2. For example, the pH value around the column can become close to 5.6 to 8.2 by flowing the animal extract with a pH value in the range between 5.6 and 8.2, preferably 6.0 and 7.0 through the column so that the adsorption of imidazole dipeptides onto the strongly acidic cation exchange resin can be enhanced while the adsorption of creatinine onto the strongly acidic cation exchange resin can be inhibited. The expression “the ratio of the positive charge of creatinine to the positive charge of imidazole dipeptides is equal to or less than 20%” means a case where the molar amount of positively charged creatinine becomes equal to or less than 0.2 when the molar amount of positively charged imidazole dipeptide is set to 1.

Other adsorption conditions such as the content of imidazole dipeptides and creatinine contained in the animal extract, the amount of animal extract loaded to the strongly acidic cation exchange resin and the adsorption temperature may be set accordingly within the range of the adsorption capacity of strongly acidic cation exchange resin since they can vary depending on the method of producing the animal extract, the salinity in the animal extract, the type of ion exchange resin and other factors. In the case of flowing the animal extract through the column packed with the strongly acidic cation exchange resin, the contact rate of the animal extract with the strongly acidic cation exchange resin is not particularly limited as long as imidazole dipeptides contained in the animal extract are adsorbed onto the strongly acidic cation exchange resin. For example, the contact rate may be a flow rate in which SV is preferably in the range between 0.5 and 8, more preferably in the range between 1 and 3.

For example, if 30 g of imidazole dipeptide is adsorbed onto 1 L of resin, the imidazole dipeptides may be adsorbed onto the strongly acidic cation exchange resin by bringing the animal extract having the amount of 3 RV to 30 RV into contact with the strongly acidic cation exchange resin at a flow rate of SV 1.0 to SV 3.0 and at a temperature of 10° C. to 60° C., preferably at room temperature (25° C.), wherein the content of imidazole peptides contained in the animal extract is equal to or more than 0.1% by mass, preferably 0.1% by mass to 1.0% by mass.

The strongly acidic cation exchange resin after brought into contact with the animal extract may adsorb contaminants contained in the animal extract. Thus, it is preferable to subject the strongly acidic cation exchange resin after brought into contact with the animal extract to a washing treatment with the use of a solvent such as water in order to get rid of such contaminants. The condition for the washing treatment is not particularly limited. For example, in the case where the animal extract is flowed through the column packed with the strongly acidic cation exchange resin, the strongly acidic cation exchange resin may be washed by flowing 0.5 RV to 3 RV of water at 10° C. to 60° C., preferably at 25° C. through the resin.

### [Step (2): Elution Treatment Step]

In Step (2), the high purity imidazole dipeptides are obtained by subjecting the imidazole dipeptides adsorbed onto the strongly acidic cation exchange resin to an elution treatment with the use of an aqueous alkaline solution and at a predetermined pH value.

By applying Step (1), the amount adsorbed onto the strongly acidic cation exchange resin is high for imidazole dipeptides and low for creatinine. Thus, if Step (2) is carried out under the condition suitable for eluting imidazole dipeptides, the high purity imidazole dipeptides with a decreased content of creatinine can be obtained.

The aqueous alkaline solution used in the elution treatment is not particularly limited in terms of the type, the concentration and the amount used, as long as it can elute the imidazole dipeptides from the strongly acidic cation exchange resin, i.e., it can render pH around the strongly acidic cation exchange resin a pH value at which the effective charge of imidazole dipeptides becomes equal to or less than zero. The aqueous alkaline solution may be selected accordingly depending on various conditions such as the type and amount of the strongly acidic cation exchange resin, the type and volume of the column, tank or other container to be packed with or to contain the strongly acidic cation exchange resin, the type and amount of imidazole dipeptide adsorbed.

Specific examples of the aqueous alkaline solution include aqueous inorganic alkaline solutions such as an aqueous sodium hydroxide solution, an aqueous potassium hydroxide solution and an aqueous ammonia solution. Since the resin can be converted to an alkali metal salt type in parallel with the elution of imidazole dipeptides, the aqueous alkaline metal hydrate solution is preferably employed. As specific examples of the aqueous alkaline metal hydrate solution, employed is more preferably a aqueous sodium hydroxide solution, still more preferably a 0.1 N to 1.0 N aqueous sodium hydroxide solution, and still even more preferably a 0.3 N to 0.5 N aqueous sodium hydroxide solution for the purpose of converting the strongly acidic cation exchange resin to the form of Na type. The use of the aqueous sodium hydroxide solution tends to enhance the elution efficiency and the recovery of imidazole dipeptides due to the difference in alkalinity as compared to the use of ammonia water.

According to the present inventors, the isoelectric point (pI) of carnosine and anserine is 8.3 and pK₂ is from 9.6 to 9.8. Therefore, it is preferable to use an aqueous alkaline solution that makes pH around the strongly acidic cation exchange resin 8.5 or more, preferably 8.5 to 15.0. For example, when the animal extract with a pH value of around 6 is flowed through the column packed with 500 L of strongly acidic cation exchange resin, and then 500 L to 1,000 L of 0.3N to 0.5N aqueous sodium hydroxide solution (1RV to 3RV) is flowed through the column at the flow rate of SV 1.0 to 3.0 at room temperature, the pH value in the column may become about 9.0 to 12.0, and may be able to efficiently elute the imidazole dipeptides adsorbed onto the strongly acidic cation exchange resin.

If the elution treatment is carried out by adding the aqueous alkaline solution to the strongly acidic cation exchange resin while stirring, the pH value around the strongly acidic cation exchange resin can become 8.5 or more uniformly and promptly without any excessive workload even if the amount of resin is large, resulting in the more effective elution of imidazole dipeptides. For example, the strongly acidic cation exchange resin packed with and held in the column may be stirred by an agitator, by blowing a gas into the column, or by gradually adding an aqueous alkaline solution.

By carrying out Steps (1) and (2), obtained are high purity imidazole dipeptides in which creatinine contaminated is reduced relative to the imidazole dipeptides. The contents of imidazole dipeptides and creatinine in the high purity imidazole dipeptides are not particularly limited as long as the high purity imidazole dipeptides are obtained by carrying out Steps (1) and (2). For example, if a hot water extract of chicken breast meat is employed as the animal extract, the content of imidazole dipeptides is, relative to dry mass (solids) of the high purity imidazole dipeptides, equal to or more than 70% by mass, preferably equal to or more than 80% by mass; and the content of creatinine is, relative to mass of imidazole dipeptides, equal to or less than 10% by mass, preferably equal to or less than 5% by mass. The upper limit of the content of imidazole dipeptides and the lower limit of the content of creatinine contained in the high purity imidazole dipeptides are not particularly limited, but are typically 100% by mass and 0% by mass, respectively. The contents of imidazole dipeptides and creatinine are determined according to the methods described in Examples below.

The high purity imidazole dipeptides obtained through Steps (1) and (2) is preferably subjected to any treatments such as a pH adjustment treatment, a decolorization treatment, a deodorization treatment, a solid-liquid separation treatment, a demineralization treatment, a concentration treatment and an aseptic treatment, for the use as a food material. Examples of such treatments include, for example, a pH adjustment treatment in which the high purity imidazole dipeptides obtained in Step (2) are adjusted to pH 6 to 8, preferably around 7 with the use of an acid such as hydrochloric acid; a decolorization and/or deodorization treatment in which a material capable of adsorbing colored and/or odorous components, such as an activated carbon and a strongly basic ion exchange resin, is used; a solid-liquid separation treatment such as a filtration treatment using a ceramic filter; a demineralization treatment using an electrodialysis membrane or a nanofiltration membrane; a concentration treatment using a evaporator; an aseptic treatment using a membrane filter; and a combination of two or more of the above treatments to be subjected in turn. While each treatment is not particularly limited in terms of conditions and procedures as long as the loss of the imidazole dipeptides does not become more significant, known methods may be employed.

For example, the demineralization treatment of high purity imidazole dipeptides may be performed at a pH value of 8.0 or less using a nanofiltration membrane with a fractional molecular weight of 500 or less and/or a sodium chloride rejection rate (the rate at which sodium chloride is retained on the membrane) of 50% or less. Such a nanofiltration membrane is described in Table 3 of Patent Document 3. For example, when the high purity imidazole dipeptides are subjected to a demineralization treatment, the salt concentration after the demineralization treatment is, as mass of sodium relative to mass of imidazole dipeptides, preferably less than or equal to 5% by mass, and more preferably less than or equal to 2% by mass.

The method according to one embodiment of the present invention may include various steps and operations before, after, or during the above steps as long as it can solve the problems of the present invention. In addition, it is preferable that the method according to one embodiment of the present invention consists of, as an ion exchange treatment, (1) subjecting an animal extract containing imidazole dipeptides and creatinine to an ion adsorption treatment in which the animal extract is brought into contact with a strongly acidic cation exchange resin having a alkali metal salt type of ion exchange group at a pH value which causes the imidazole dipeptides to become positively charged and causes the ratio of the positive charge of creatinine to the positive charge of imidazole dipeptides to become equal to or less than 20%, and then subjecting the strongly acidic cation exchange resin to a washing treatment with the use of water to adsorb the imidazole dipeptides onto the strongly acidic cation exchange resin; and (2) subjecting the strongly acidic cation exchange resin adsorbing the imidazole dipeptides to an elution treatment with the use of an aqueous alkaline solution and at a pH value which causes the imidazole dipeptides to become charged at zero or negatively charged to obtain high purity imidazole dipeptides.

While a Specific embodiment of the method of the present invention will be described below, the method of the present invention is not limited to it.

An acid is passed through a column packed with a strongly acidic cation exchange resin to convert the ion exchange group of the resin to the H type one, and then water is passed through the column, and further an aqueous alkali metal salt solution is passed through the column to convert the ion exchange group of the resin to the Na type one. Subsequently, the excess aqueous alkali metal salt solution is washed away by flowing water through the column.

The animal parts containing imidazole dipeptides and creatinine are added to water, and the resulting mixture is subjected to a hot water extraction treatment at a temperature in the range between 80° C. and 95° C. for tens of minutes to several hours. The obtained hot water extract is subjected as it stands or after subjected to a demineralization treatment using an electrodialysis or nanofiltration membrane, to a concentration treatment and a solid-liquid separation treatment, thereby obtaining an animal extract in which imidazole dipeptides are 0.1% by mass to 1.0% by mass, Brix is 1.0% to 10.0%, and pH is 5.6 to 8.0.

The animal extract is flowed through the column packed with the Na type strongly acidic cation exchange resin with 3 RV to 30 RV and at SV 1 to SV 3, and then water is flowed through the column with RV 0.5 to RV 2.0 to adsorb imidazole dipeptides contained in the animal extract onto the strongly acidic cation exchange resin. The pH value in the column after this adsorption treatment is in the range between 5.6 and 8.0.

Subsequently, 0.1 N to 1.0 N aqueous alkali metal salt hydroxide solution is flowed through the column at SV 1.0 to SV 3.0 and with 1.0 RV to 2.0 RV to obtain high purity imidazole dipeptide as an eluate. The pH value in the column after the elution treatment is in the range between 8.5 and 14.0.

The eluate is sequentially subjected to a pH adjustment treatment that adjusts it to near neutrality with the use of an acid, a demineralization treatment using an electrodialysis membrane or a nanofiltration membrane, a concentration treatment using an evaporator, and a sterile filtration treatment using a membrane filter with a pore size of 0.20 μm to 0.45 μm, to obtain an imidazole dipeptide product.

The dosage form of the high purity imidazole dipeptides obtained by the method according to one embodiment of the present invention is not particularly limited and may be in either liquid form or solid form. In order to make them suitable for long-term storage, it is preferable to render the high purity imidazole dipeptides in the liquid form those in the powder form by subjecting them to a drying treatment such as an air-drying, a decompression drying, a freeze drying or a spray drying.

The uses of high purity imidazole dipeptides obtained by the method according to one embodiment of the present invention are not particularly limited. The high purity imidazole dipeptides have a large content of imidazole dipeptides and a small content of creatinine. In addition, the high purity imidazole dipeptides may be demineralized, decolorized and/or deodorized. Thus, the high purity imidazole dipeptides may be used as raw materials for various compositions including oral compositions such as foods, drinks and pharmaceuticals, and topical compositions such as cosmetics, or as the compositions themselves, in anticipation of the biological activities such as anti-fatigue and hypoglycemic effects possessed by imidazole dipeptides.

While the content of high purity imidazole dipeptides in a food, a drink and a cosmetic is not particularly limited, it is, for example, an amount determined in such a way that the amount of imidazole dipeptides becomes, as a dry mass relative to the total amount of the food, the drink or the cosmetic, preferably 0.001% by mass or more, and more preferably from 0.1% by mass to 99% by mass.

The dosage form of the food and the drink is not particularly limited, but includes, for example, the forms of liquid, powder, tablet, round, fine grain, granule, capsule, jelly, chewable and paste.

Specific examples of foods and drinks may include, but not limited to, the followings: drinks, such as soft drinks, carbonated drinks, fruit drinks, vegetable juices, lactic acid bacteria drinks, milk drinks, soy milk, mineral water, tea drinks, coffee drinks, sports drinks, alcoholic drinks and jelly drinks; vegetable processed products such as tomato puree, canned mushrooms, dried vegetables and pickles; fruits processed products such as dried fruits, jams, fruit purees and canned fruits; spices such as curry powder, horseradish, ginger, spice blends and seasoning powders; noodles (including fresh and dried noodles) such as pasta, udon, soba noodles, ramen noodles, and macaroni; breads such as breads, sweet breads, prepared breads and doughnuts; flour products such as aliphatized rice, oatmeal, fu and batter flour; confectionery such as baked cakes, cookies, rice cakes, candies, chocolates, chewing gums, snack confectionery, chilled desserts, candied confectionery, Japanese cakes, western cakes, semi-baked cakes, pudding and ice cream; bean products such as azuki beans, tofu, natto, soybean flour, yuba (bean curd lees), cooked beans and peanuts; processed foods such as honey and royal jelly; meat products such as ham, sausage and bacon; dairy products such as yogurt, pudding, condensed milk, cheese, fermented milk, butter and ice cream; egg processed products; fish processed foods such as dried fish, kamaboko, chikuwa and fish sausage; processed seaweed such as dried seaweed, kelp and tsukudani; fish egg processed products such as cod roe, herring roe, salmon roe and karasumi; seasonings such as dashi broth, soy sauce, vinegar, mirin, consomme base, Chinese base, concentrated dashi, dressing, mayonnaise, ketchup and miso; edible fats and oils such as salad oil, sesame oil, linoleum oil and diacylglycerol; benibana oil, etc.; prepared foods such as soups (including powders and liquids), cooked food, retort food, chilled food and semi-cooked food (e.g., cooked rice stock, crab ball stock).

When the high purity imidazole dipeptides according to one embodiment of the present invention is used to be blended into a cosmetic, the cosmetic may be used in the various forms such as lotion, emulsion, cream, gel and pack.

Now, the present invention will be described in greater detail by way of examples. Note, however, that the present invention is by no means limited by those examples and may be realized in various different modes as long as such modes can dissolve the problems to be solved by the present invention.

### EXAMPLES

### Example 1: Evaluation of Adsorption Behavior of Imidazole Dipeptides and Creatinine on Strongly Acidic Cation Exchange Resin

In view of the difference between the adsorption behaviors of imidazole dipeptides, including anserine and carnosine, and creatinine on a strongly acidic cation exchange resin, each electrical dissociation behavior of the compounds with respect to pH values was experimentally measured by the titration method as mentioned below. The terms “pK” and “pI” are the dissociation constant and the isoelectric point, respectively.

The 0.1 M aqueous solutions of L-anserine, L-carnosine and creatinine were prepared and the titration curves were generated with 0.1 N sulfuric acid and 0.1 N sodium hydroxide according to known methods. From each titration curve, pK₁, pK_{R} and pK₂ were determined. The results are shown in Table 1.

TABLE 1

| Type | pK₁ | pK_{R} | pK₂ | pI |
|---|---|---|---|---|
| Anserine | 2.8 | 7.0 | 9.6 | 8.3 |
| Carnosine | 2.8 | 6.8 | 9.8 | 8.3 |
| Creatinine | — | 4.9 | — | — |

In Table 1, with respect to anserine and carnosine, pK₁ represents the dissociation of carboxyl group, pK_{R} represents the dissociation of imidazole group, and pK₂ represents the dissociation of amino group. With respect to creatinine, pK_{R} represents the dissociation of imidazole group.

Based on the measured results, the state of the charge of anserine, carnosine and creatinine at each pH value is shown in FIG. 1. FIG. 1 shows that the imidazole dipeptides, anserine and carnosine, had a 1-valent positive charge at pH 5, a 0.5-valent positive charge at pH 7, and a 0.1-valent positive charge at pH 8, and thus can be adsorbed on the cation exchange resin at the respective pH values. On the other hand, FIG. 1 also shows that creatinine had a 0.5-valent positive charge at pH 5, a 0.2-valent positive charge at pH 5.6, and a 0.1-valent positive charge at pH 6, but creatinine had no positive charge when the pH value was equal to or more than 7.

These results are well consistent with the data disclosed in the references as published so far. For example, Bate-Smith (Bate-Smith, E. C., J. Physiol. (London) 92, 336 (1938)) discloses the results that pK₂ of carnosine was 6.83, and Doutsch et al. (Dutch, A., Eggleton, P., Biochem. J. 32, 209 (1938)), discloses the results that pK₂ of anserin was 7.04. In these references, pK₂ means the dissociation constant of imidazole group and corresponds to pK_{R} in Table 1. In addition, Eadie et al. (Geoge S. Eadie and Andrew Hunter, J. Biol. Chem. 1926, 67:237-244) states that pK_{b} of creatinine was 9.20. If converted to pK_{R}, it is 4.8. Thus, each pK_{R} for anserine, carnosine and creatinine in Table 1 was well consistent with the data described in the references.

On the basis of the above results, verified was the pH value at which the imidazole dipeptides can be adsorbed but creatinine cannot be adsorbed, focusing on the difference between the dissociation constant of anserine and carnosine and that of creatinine. FIG. 2 shows the results in which each ratio of the positive charge of creatinine to the positive charge of imidazole dipeptides was plotted with respect to each pH value. As shown in FIG. 2, the ratio of the positive charge of creatinine to the positive charge of imidazole dipeptides in the range between pH 5.6 and pH 8.2 was equal to or less than 0.2. Thus, it was found that within the above pH range, the imidazole dipeptides could be more adsorbed on the strongly acidic cation exchange resin than creatinine. From the above results, the present inventors found the possibilities of separating imidazole dipeptides from creatinine based on the difference in pH at adsorption equilibrium between them during the adsorption onto a strongly acidic cation exchange resin.

### Example 2: Batch Adsorption Evaluation in the Coexistence of Imidazole Dipeptides and Creatinine

In reference to the results obtained in Example 1, the following adsorption tests in batches were performed in the coexistence of imidazole dipeptides and creatinine at the stages of pH 4 to pH 8.5. From now, among imidazole dipeptides, a mixture of anserine and carnosine may be referred to as “AC,” and creatinine may be referred to as “Cre.”

The chicken extract obtained by subjecting chicken breast meat to the hot water extraction treatment was clarified by diatomaceous earth filtration and diluted with water to prepare a filtrate containing 0.56% by mass of imidazole dipeptides and 0.21% by mass of creatinine. Hydrochloric acid or sodium hydroxide was added to the filtrate to prepare an aqueous chicken extract solution in stages at 25° C. in the range between pH 3 and pH 9. To a 100-mL beaker containing 10 ml of strongly acidic cation exchange resin (“DIAION SK1B”; manufactured by Mitsubishi Chemical), which was converted and equilibrated to the form of Na type in advance, the aqueous chicken extract solution was added in such a way that the amount of imidazole dipeptides added became 0.20 g. Then, water was further added to the beaker to make 100 ml. The solution in the beaker was stirred with a magnetic stirrer for 2 hours at 25° C. and then subjected to centrifugation. The resulting supernatant was analyzed by GPC-HPCL to determine the amounts of imidazole dipeptides and creatinine adsorbed with the use of the aqueous chicken extract solutions with each pH value. Here, each amount of imidazole dipeptides and creatinine adsorbed onto the resin was determined from the difference between the amount loaded and the amount contained in the non-adsorbed solution (supernatant). With GPC-HPLC, “TSKgel 2500 PWXL (particle size: 6 μm, diameter: 7.8 mm×length: 300 mm)” (manufactured by Tosoh) was employed as the column and a 0.1% trifluoroacetic acid-added 45% acetonitrile was employed as the developing solvent. HPLC, “PU-2089” (manufactured by Nippon Spectroscope; flow rate: 0.5 ml/min, detector wavelength: 210 nm) was employed.

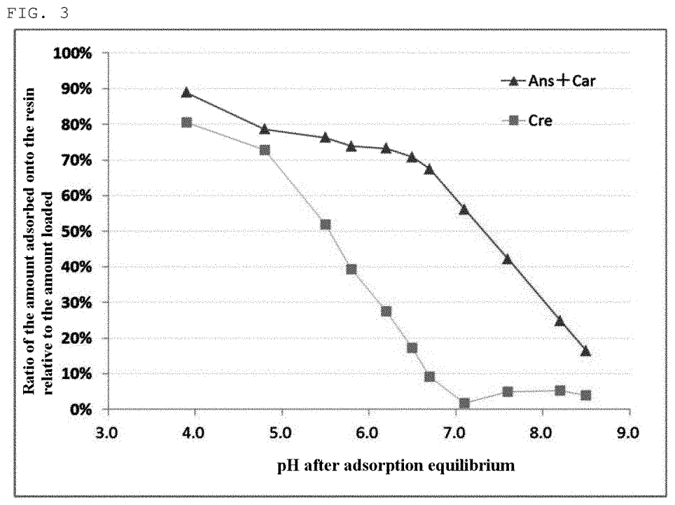

The measured results are shown in FIG. 3. As shown in FIG. 3, the amount of creatinine adsorbed decreased as the pH value increased from 4 to 6.5, and thereafter the adsorption was almost completely absent from pH 7 to 8.5. On the other hand, the amount of imidazole dipeptides adsorbed did not almost decrease between pH 4 and 6.5, and after pH 6.5 while the amount adsorbed decreased as the pH value increased, the imidazole dipeptides were adsorbed until the pH value reached around pH 8.5.

From the above results, it was found that by using the Na type strongly acidic cation exchange resin, imidazole dipeptides could be preferentially adsorbed over creatinine in the range between weakly acidic and weakly alkaline conditions, i.e., around between pH 5.6 and 8.2.

### Example 3. Method of Producing Composition Containing Imidazole Dipeptides (1)

The column was packed with 500 L of strongly acidic cation exchange resin (“DIAION SK1B”; manufactured by Mitsubishi Chemical). Through the column packed with the resin, 2 RV of 1N hydrochloric acid was flowed to convert the resin in the column to the form of H type, and then 1 RV of RO water was flowed. Then, 2 RV of 1N sodium hydroxide and 1 RV of RO water were flowed through the column in sequence to convert the resin in the column to the form of Na type. The pH value in the column was around between 10 and 11.

To 2,000 kg of chicken breast meat (containing about 15 kg of imidazole dipeptides), 3,000 kg of municipal water was added, and the mixture was subjected to the hot water extraction treatment at 90° C. for 60 minutes, and the resultant was then concentrated by decompression using an evaporator to obtain a crude chicken extract in which the content of imidazole dipeptides was 0.47% by mass relative to the total mass, and the content of creatinine was 30% relative to mass of imidazole dipeptides (30% by mass). The crude chicken extract obtained was concentrated by decompression without a pH adjustment treatment in such a way that Brix reached 5.8% and the content of imidazole dipeptides reached 0.6% by mass, and the resulting concentrate was then subjected to a diatomaceous earth filtration treatment to obtain a chicken extract. The pH value of the chicken extract was about 6.2.

The obtained chicken extract was subjected to a adsorption treatment by flowing the chicken extract through the Na type resin-packed column at 4.5 RV and SV 2.0, and then passing 1 RV of RO water through the column. The pH value in the column was around between 7.5 and 8 after the adsorption treatment.

Then, 0.4 N sodium hydroxide was passed through the column at SV 2.0 and 1.5 RV to obtain high purity imidazole dipeptides as an eluate. The pH value in the column was around between 8.5 and 12.0 after the elution treatment. In the obtained high purity imidazole dipeptides, the content of imidazole dipeptides was about 80% by mass relative to the total mass, and the content of creatinine was 5% by mass relative to mass of imidazole dipeptides.

The eluate (750 L) was adjusted to become around pH 7.0 with hydrochloric acid, decolorized with an activated charcoal, and then filtered through a ceramic filter. The resulting filtrate was subjected to demineralization and concentration treatments using the nanofiltration membrane (“DRA-4510”; manufactured by Daicen Membrane Systems; rejection rate of sodium chloride: 45%; filtration membrane area: approximately 7.5 m²), and then aseptically filtered with the use of a membrane filter having pore sizes of 0.45 μm to obtain 150 kg of liquid product containing 10% by mass imidazole dipeptides.

The content of imidazole dipeptides in the obtained imidazole dipeptides product was about 80% by mass per dry mass of the product; the content of creatinine was 2% by mass per dry mass of imidazole dipeptides; and the content of salt (as a sodium amount) was about 1% by mass per dry mass of imidazole dipeptides.

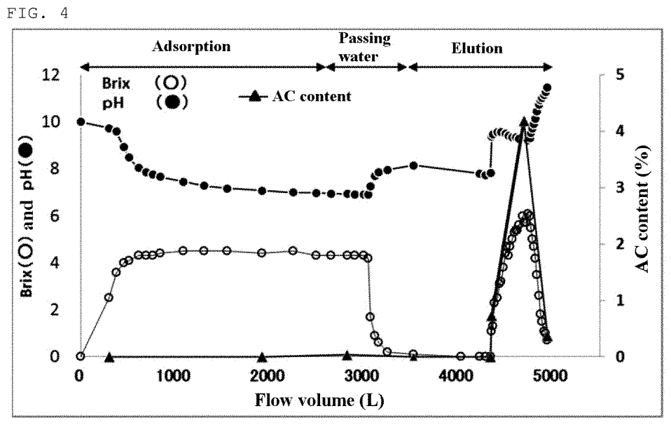

As a summary of the above results, FIG. 4 shows the changes in pH, Brix and imidazole dipeptides levels in relation to the amount of chicken extract flowed during the adsorption and elution treatments.

### Example 4. Purity Evaluation of Imidazole Dipeptides

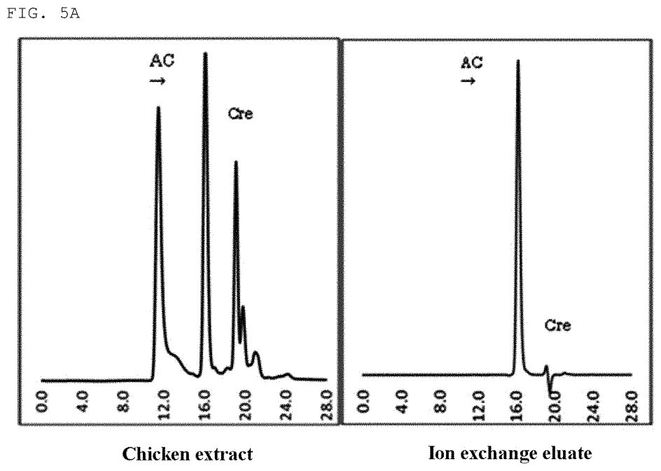

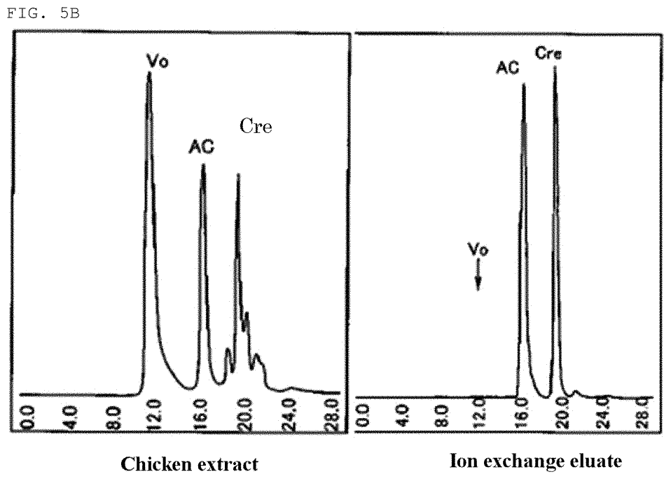

Using the chicken extract and eluate (ion exchange eluate) of Example 3, each amount of anserine, carnosine and creatinine was determined by GPC-HPLC. The results are shown in FIG. 5A. As reference examples, FIG. 5B shows FIG. 1 and FIG. 2 as described in Patent Document 3. The summary of these results is also shown in Table 2.

TABLE 2

| | Ratio of the area of creatinine to | |
|---|---|---|
| | the area of imidazole dipeptides | |
| | Cre/(Ans + Car) | |
| | Method of | Method of Patent |
| Sample | Example 3 | Document 3 |
| Chicken extract | 0.81 | 0.92 |
| Ion exchange eluate | 0.04 | 1.06 |
| Ion exchange eluate/ | 0.049 | 1.152 |
| chicken extract | | |

As shown in FIG. 5A and FIG. 5B, and Table 2, it was confirmed that the method of Example 3 removed most of creatinine by the ion exchange treatment, and that the ratio of creatinine to imidazole dipeptides in the ion exchange eluate was very low when compared to the ratio in the raw material.

From the above results, it was found that the present method could produce high purity imidazole dipeptides with a low content of creatinine on an industrial scale. Therefore, it was found that the present method was an excellent method for obtaining large quantities of high purity imidazole dipeptides.

### Example 5. Method of Producing Composition Containing Imidazole Dipeptides (2)

To 2,500 kg of white salmon with its head and organs removed, which contains about 12 kg of imidazole dipeptides, 3,000 kg of city water was added, and the mixture was subjected to a hot water extraction treatment at 90° C. for 20 minutes, resulting in a crude salmon extract containing 3.2% of Brix, 0.35% by mass of imidazole dipeptides relative to the total mass, and 20% (20% by mass) of creatinine relative to mass of imidazole dipeptides. The resulting crude salmon extract was filtered through diatomaceous earth without a pH adjustment treatment to obtain a salmon extract. The pH value of the salmon extract was 6.0.

The obtained salmon extract was subjected to a adsorption treatment by flowing the extract through the Na type resin-packed column at 6.5 RV and SV 2.0, and then passing 1 RV of RO water through the column. The pH value in the column after the adsorption treatment was in the range between around 7.5 and 8.

The column was then subjected to the same elution treatment as in Example 3 to obtain high purity imidazole dipeptides as the eluate. The pH value in the column after the elution treatment was in the range between around 8.5 and 12.0. In the obtained high purity imidazole dipeptides, the content of imidazole dipeptides was about 75% by mass, and the content of creatinine was 7% by mass relative to imidazole dipeptides.

The resulting eluate was subjected as in Example 3 to the pH adjustment treatment, the decolorization treatment using the activated charcoal, the filtration treatment using the ceramic filter, the demineralization treatment using the nanofiltration membrane, and the aseptic filtration treatment using the membrane filter in sequence to obtain 100 kg of liquid product containing 10% by mass of imidazole dipeptides.

In the obtained imidazole dipeptides product, the content of imidazole dipeptides was about 75% by mass per dry mass of the product; the content of creatinine was 5% by mass per dry mass of imidazole dipeptides; and the content of salt as mass of sodium was about 1% by mass per dry mass of imidazole dipeptides.

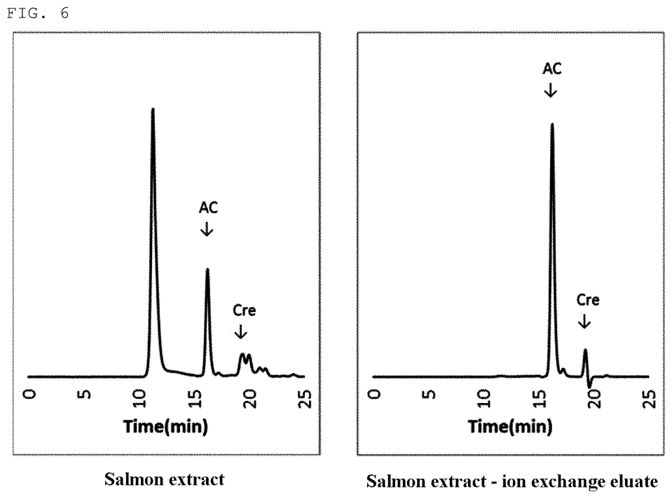

In addition, as in Example 4, each amount of anserine, carnosine and creatinine in the salmon extract and the eluate (ion exchange eluate) was determined by GPC-HPLC. The results are shown in FIG. 6. As shown in FIG. 6, it was confirmed with respect to the present method that the ion exchange treatment removed most of creatinine, and the ratio of creatinine to imidazole dipeptides in the ion exchange eluate was very low when compared to the ratio in the raw material.

### INDUSTRIAL APPLICABILITY

The present invention is useful in the fields of foods and beverages, pharmaceuticals, cosmetics, quasi-pharmaceutical products and the like, and particularly has advantage in being capable of producing anti-fatigue compositions, antihyperglycemic compositions or raw materials for these compositions.

We claim:

1. A method of producing high purity imidazole dipeptides, comprising:
(1) subjecting an animal extract containing imidazole dipeptides and creatinine to an ion adsorption treatment in which the animal extract is brought into contact with a strongly acidic cation exchange resin at a pH value so that the imidazole dipeptides are adsorbed onto the strongly acidic cation exchange resin, wherein the pH value is a pH value at which the imidazole dipeptides become positively charged and the ratio of the positive charge of creatinine to the positive charge of imidazole dipeptides becomes equal to or less than 20%, and the strongly acidic cation exchange resin has an alkali metal salt type of ion exchange group; and
(2) subjecting the strongly acidic cation exchange resin adsorbing the imidazole dipeptides to an elution treatment with the use of an aqueous alkaline solution and at a pH value which enables the imidazole dipeptides to become charged at zero or negatively charged to obtain high purity imidazole dipeptides.

2. The method according to claim 1, wherein the step (1) is subjecting an animal extract containing imidazole dipeptides and creatinine to an ion adsorption treatment in which the animal extract is brought into contact with a strongly acidic cation exchange resin at a pH value followed by subjecting the strongly acidic cation exchange resin to a washing treatment using water so that the imidazole dipeptides are adsorbed onto the strongly acidic cation exchange resin, wherein the pH value is a pH value at which the imidazole dipeptides become positively charged and the ratio of the positive charge of creatinine to the positive charge of imidazole dipeptides becomes equal to or less than 20%, and the strongly acidic cation exchange resin has an alkali metal salt type of ion exchange group.

3. The method according to claim 1, wherein in the step (1), the pH value is in the range between 5.6 and 8.2, and/or in the step (2), the pH value is in the range between 8.5 and 15.0.

4. The method according to claim 1, wherein the strongly acidic cation exchange resin is a strongly acidic cation exchange resin having an ion exchange group converted to an alkali metal salt type by means of passing an aqueous acid solution and an aqueous alkali metal salt solution in sequence through the resin.

5. The method according to claim 1, wherein the aqueous alkaline solution is an aqueous alkali metal hydrate solution.

6. The method according to claim 1, wherein the aqueous alkaline solution is an aqueous sodium hydroxide solution.

7. The method according to claim 1, wherein the alkali metal salt is at least one alkali metal salt selected from the group consisting of sodium and potassium.

8. The method according to claim 1, wherein the animal extract is an animal extract subjected to a demineralization treatment.

9. The method according to claim 1, wherein the animal extract is at least one animal extract selected from the group consisting of chicken extract, bovine extract, pig extract, salmon extract, bonito extract and tuna extract.
